(19) United States
(12) Patent Application Publication
(10) Pub. No.: US 2026/0021059 A1
(43) Pub. Date: Jan. 22, 2026

(54) THERAPEUTIC COMBINATIONS AND METHODS

(71) Applicant: RUTGERS, THE STATE UNIVERSITY OF NEW JERSEY, New Brunswick, NJ (US)

(72) Inventor: Morgan JAMES, New Brunswick, NJ (US)

(73) Assignee: RUTGERS, THE STATE UNIVERSITY OF NEW JERSEY, New Brunswick, NJ (US)

(21) Appl. No.: 18/996,728

(22) PCT Filed: Jul. 17, 2023
(86) PCT No.: PCT/US2023/027918

§ 371 Date: Jan. 17, 2025

Related U.S. Application Data
(60) Provisional application No. 63/390,584, filed on Jul. 19, 2022.

(51) Int. Cl. A61K 31/137; A61K 31/4375; A61K 31/551; A61P 3/04

(52) U.S. Cl. CPC A61K 31/137; A61K 31/4375; A61K 31/551; A61P 3/04

(57) ABSTRACT

The invention provides methods and compositions for treating food related disorders, as well as methods for enhancing the therapeutic activity of a stimulant and methods for lowering the amount of a stimulant required to achieve a therapeutic result in a human.

### PRIORITY

This application claims priority to U.S. Provisional Patent Application No. 63/390,584, filed 19 Jul. 2022. The entire contents of this United States Provisional Patent Application is hereby incorporated herein by reference.

### STATEMENT REGARDING FEDERALLY SPONSORED RESEARCH

This invention was made with government support under DA045765 awarded by the National Institute on Drug Abuse. The government has certain rights in the invention.

### BACKGROUND OF THE INVENTION

Binge eating disorder (BED) is characterized by repeated episodes of excessive pathological, non-homeostatic food consumption (American Psychiatric A, American Psychiatric A, Force DSMT. Diagnostic and statistical manual of mental disorders: DSM-5. 2013). These binge episodes occur in repeated, discrete time periods, and are accompanied by a perceived loss of control over how much is consumed. BED is highly comorbid with obesity—an estimated 5-15% of obese people have BED, and individuals with BED are 3-6 times more likely to be obese than individuals without an eating disorder (McCuen-Wurst C, et al., Ann N Y Acad Sci. 2018; 1411:96-105). Psychological treatments such as cognitive behavioral therapy and interpersonal therapy are generally considered the first line of treatment for BED (Ghaderi A, et al., Peer J. 2018; 6:e5113; and Wilson G T, et al., Arch Gen Psychiatry. 2010; 67:94-101) although the efficacy of these approaches is limited (Linardon J. Int J Eat Disord. 2018; 51:785-97). Presently, only one medication, lisdexamfetamine (LDX), a d-amphetamine prodrug, has gained regulatory approval specifically for the treatment of moderate to severe BED in adults (Appolinario J C, et al., Expert Opin Invest Drugs. 2019; 28:1081-1094). Although daily LDX administration is highly effective at reducing binge frequency, only about 35-50% of patients report complete binge cessation, leaving the majority of BED patients with ongoing symptomology (McElroy S L, et al., JAMA Psychiatry. 2015; 72:235-46; McElroy S L, et al., Neuropsychopharmacology. 2016; 41:1251-60; and Hudson J I, et al., JAMA Psychiatry. 2017; 74:903-10). Currently there is a need for improved compositions and methods that can be used to treat BED and associated conditions.

### SUMMARY OF THE INVENTION

Applicant has identified novel combinations and therapeutic methods that can be used to treat food related disorders. The combinations and therapeutic methods reduce the amount of amphetamine needed to treat the disorders and/or reduce the unwanted side effects (e.g. sleep disturbance or risk of abuse) associated with amphetamine treatment. The reduction in sleep disturbance abuse liability, or other unwanted side effects results not only from the direct activity of the orexin receptor antagonist, but also from the lower dose of amphetamine required to achieve the desired therapeutic effect when the amphetamine is administered in combination with the orexin receptor antagonist.

Accordingly, in one embodiment, the invention provides a method for treating a food related disorder in a human comprising administering a reduced amount of an amphetamine (e.g. less than about 0.40 mg/kg) and an orexin receptor antagonist to the human.

In another embodiment, the invention provides a method for treating a binge eating disorder in a human, comprising administering, once daily, less than about 0.40 mg/kg of an amphetamine and less than about 10 mg suvorexant to the human, wherein the administration of the amphetamine and the suvorexant results in less sleep disruption, abuse liability or other unwanted side effects than administration of a 0.40 mg/kg dose of amphetamine alone.

In another embodiment, the invention provides a method comprising, reducing the abuse liability, wake promoting, or other unwanted side effects associated with administration of a stimulant (e.g. amphetamine, methylphenidate, dexamethylphenidate, modafinil, or benzphetamine) to an animal, comprising administering an orexin receptor antagonist to the animal.

In another embodiment, the invention provides a method comprising, treating a disease (e.g., attention-deficit/hyperactivity disorder, ADHD; attention deficit disorder, ADD) that is treatable with a stimulant (e.g. amphetamine, methylphenidate, dexamethylphenidate, modafinil, or benzphetamine) in an animal by administering to the animal, the stimulant and an orexin receptor antagonist that reduces the abuse liability, wake promoting, or other unwanted side effects of the stimulant.

In another embodiment, the invention provides a pharmaceutical composition for once daily dosing for the treatment of a food related disorder in a human, comprising less than about 40 mg of an amphetamine, an orexin receptor antagonist, and a pharmaceutically acceptable carrier.

In another embodiment, the invention provides a pharmaceutical composition for once daily dosing for the treatment of binge eating disorder in a human, comprising less than about 3 mg of amphetamine, less than about 5 mg suvorexant, and a pharmaceutically acceptable carrier.

In another embodiment, the invention provides a method for lowering the amount of an amphetamine required to achieve a therapeutic result in a human, comprising, administering the amphetamine to the human in combination with an orexin receptor antagonist

In another embodiment, the invention provides a method for enhancing the therapeutic activity of an amphetamine in a human, comprising, administering the amphetamine to the human in combination with an orexin receptor antagonist.

In another embodiment, the invention provides the use of less than about 0.40 mg/kg of amphetamine to treat a food related disorder in a human in combination with an orexin receptor antagonist

In another embodiment, the invention provides the use of less than about 0.40 mg/kg of amphetamine daily to treat a binge eating disorder in combination with less than about 10 mg suvorexant daily, wherein the administration of the amphetamine and the suvorexant results in less insomnia than administration of a 0.40 mg/kg dose of amphetamine alone.

In another embodiment, the invention provides the use of an orexin receptor antagonist to lower the amount of an amphetamine required to achieve a therapeutic result in a human.

In another embodiment, the invention provides the use of an orexin receptor antagonist to enhance the therapeutic activity of an amphetamine in a human.

In another embodiment, the invention provides the use of less than about 0.40 mg/kg of amphetamine to prepare a medicament that is useful to treat a food related disorder in a human in combination with an orexin receptor antagonist.

In another embodiment, the invention provides the use of less than about 0.75 mg/kg of amphetamine daily to prepare a medicament that is useful to treat a binge eating disorder in combination with less than about 10 mg suvorexant daily, wherein the administration of the amphetamine and the suvorexant results in less insomnia than administration of a 0.75 mg/kg dose of amphetamine alone.

In another embodiment, the invention provides the use of less than about 0.40 mg/kg of amphetamine daily to prepare a medicament that is useful to treat a binge eating disorder in combination with less than about 10 mg suvorexant daily, wherein the administration of the amphetamine and the suvorexant results in less insomnia than administration of a 0.40 mg/kg dose of amphetamine alone.

In another embodiment, the invention provides the use of an orexin receptor antagonist to prepare a medicament that is useful to lower the amount of an amphetamine required to achieve a therapeutic result in a human.

In another embodiment, the invention provides the use of an orexin receptor antagonist to prepare a medicament that is useful to enhance the therapeutic activity of an amphetamine in a human.

In another embodiment, the invention provides the use of an orexin receptor antagonist to reduce the abuse liability, wake promoting, or other unwanted side effects associated with administration of a stimulant (e.g. amphetamine, methylphenidate, dexamethylphenidate, modafnil or benzphetamine) to an animal.

In another embodiment, the invention provides the use of an orexin receptor antagonist to treat a disease (e.g., ADHD, ADD) that is treatable with a stimulant (e.g. amphetamine, methylphenidate, dexamethylphenidate, modafnil or benzphetamine) in combination with a stimulant, where the orexin receptor antagonist reduces the abuse liability, wake promoting, or other unwanted side effects of the stimulant.

In another embodiment, the invention provides the use of an orexin receptor antagonist to prepare a medicament for reducing the abuse liability or wake promoting associated with administration of a stimulant (e.g. amphetamine, methylphenidate, dexamethylphenidate, modafnil or benzphetamine) to an animal in combination with the stimulant.

In another embodiment, the invention provides the use of an orexin receptor antagonist to prepare a medicament for treating a disease (e.g., ADHD, ADD) that is treatable with a stimulant (e.g. amphetamine, methylphenidate, dexamethylphenidate, modafnil or benzphetamine) in combination with the stimulant, where the orexin receptor antagonist reduces the abuse liability, wake promoting, or other unwanted side effects of the stimulant.

In another embodiment, the invention provides a method for lowering the amount of a stimulant required to achieve a therapeutic result in an animal, comprising, administering the stimulant to the animal in combination with an orexin receptor antagonist.

In another embodiment, the invention provides an orexin receptor antagonist to lower the amount of a stimulant required to achieve a therapeutic result in an animal.

In another embodiment, the invention provides the use of an orexin receptor antagonist to prepare a medicament to lower the amount of a stimulant required to achieve a therapeutic result in an animal in combination with the stimulant.

In another embodiment, the invention provides a method comprising, treating a disease in an animal by administering a stimulant and an orexin receptor antagonist to the animal.

In another embodiment, the invention provides the use of a stimulant and an orexin receptor antagonist for the prophylactic or therapeutic treatment of a disease (e.g., a disease that is treatable with a stimulant).

In another embodiment, the invention provides the use of an orexin receptor antagonist to prepare a medicament for treating a disease in an animal (e.g., a disease that is treatable with a stimulant) in combination with a stimulant.

In another embodiment, the invention provides a method comprising, reducing the abuse liability, insomnia, sleep fragmentation, wakefulness, or sleep disturbances associated with administration of a stimulant to an animal, comprising administering an orexin receptor antagonist to the animal.

### BRIEF DESCRIPTION OF THE DRAWINGS

FIGS. 1a-1b show a) that when administered alone at low doses, d-amphetamine (0.1875, 0.375 mg/kg; i.p.) has no effect on palatable food intake in rats. Similarly, when administered alone at low doses, suvorexant (10 mg/kg; i.p.) has no effect on palatable food intake in rats. Surprisingly, the combination of low doses of d-amphetamine (0.1875, 0.375 mg/kg) and suvorexant (10 mg/kg) suppresses palatable food intake, indicating additive effects of these compounds. RM ANOVA F (3.25, 35.73)=9.73, p<0.0001. Holm-Sidak post-hoc tests: **p<0.05. ns: p>0.05. B) shows suppression of palatable food intake can be achieved by combining a subthreshold dose of d-amphetamine (0.375 mg/kg) with very low doses of suvorexant (0.918 and 1.84 mg/kg). RM ANOVA F (1.98, 21.8)=6.41, p=0.0066, Holm-Sidak post-hoc tests: *p<0.05.

FIGS. 2a-2b show a) Treatment with a selective Ox1R antagonist SB334867 (https://pubmed.ncbi.nlm.nih.gov/11250867) dose-dependently decreased binge-like eating of a sweetened fat solution in rats. A significant reduction was observed only at a high dose of SB334867 (30 mg/kg). RM ANOVA F (1.1, 4.3)=6.68, p=0.055, Holm-Sidak post-hoc tests, *p<0.05. b) Surprisingly, the combination of subthreshold dose of d-amphetamine (0.375 mg/kg) and SB334867 (10 mg/kg) significantly reduced binge-like eating. RM ANOVA F (1.6, 6.2)=7.24, p=0.0274, Holm-Sidak post-hoc test, *p<0.05.

FIG. 3 shows that combination of d-amphetamine and suvorexant at doses that suppressed binge eating (0.375 and 1.84 mg/kg, respectively) reduced lever responding for sucrose pellets on a fixed ratio 5 (FR5) schedule, indicating a reduction in motivation for food reward. Paired t-test, t=4.442, p=0.003.

FIG. 4 shows data from Example 5. Rats were tested for baseline locomotor activity (day 0) and then pseudo-randomly divided into treatment groups. Treatment with d-amphetamine (0.375 mg/kg; i.p) promoted increased locomotor activity that increased with repeated (7 d) dosing. On the final day of treatment, locomotor reactivity was significantly lower in rats that were treated with the d-amphetamine combined with suvorexant (1.8 or 10 mg/kg), compared to d-amphetamine alone+vehicle. These data indicate that suvorexant abrogates the locomotor activating properties of d-amphetamine. Mixed model ANOVA, Treatment x time interaction F(21,189)=5.599, p<0.0001. Holm-Sidak post-hoc tests, *p<0.05, **p<0.01.

FIGS. 5a-5b show data from Example 6. Rats were tested for baseline sleep using electroencephalography (EEG) and electromyography (EMG) recordings. Time spent in active wake and rapid eye movement (REM) sleep at baseline is depicted as 100%. a) At a dose necessary to suppress binge-like eating (0.75 mg/kg), d-amphetamine administration 1 hr prior to the onset of the inactive period increased total time in active wake. In contrast, the combination of d-amphetamine and suvorexant at doses that suppressed binge eating (0.375 and 1.84 mg/kg, respectively) did not affect total time in active wake. b) Similarly, d-amphetamine alone decreased total time in REM sleep compared to baseline; the combination of d-amphetamine and suvorexant did not. *p<0.05 compared to baseline.

### DETAILED DESCRIPTION OF THE INVENTION

As use herein, the term “amphetamine” includes racemic alpha-methylphenethylamine, levoamphetamine, and dextroamphetamine as well as pharmaceutically acceptable salts thereof and prodrugs (e.g., Lisdexamfetamine Dimesylate (LDX)) and mixtures thereof and extended-release formulations. The following amphetamine compounds are commercially available (Millipore Sigma): D-Amphetamine hemisulfate salt solution, D-Amphetamine hemisulfate salt, Dexamfetamine sulfate, Dextroamphetamine sulfate, (±)-Amphetamine solution, Lisdexamfetamine dimesylate solution, S(+)-Amphetamine (dextro-Amphetamine) solution, R(−)-Amphetamine (levo-Amphetamine), and (±)-Amphetamine solution.

As used herein, the term “orexin receptor antagonist” includes selective orexin-1 receptor antagonists, selective orexin-2 receptor antagonists, and dual orexin receptor antagonists. The term includes any compound with antagonist actions at the orexin-1 receptor, orexin-2 receptor, or both.

As used herein the term “dual orexin receptor antagonist” includes compounds that have antagonist activity at both the orexin-1 receptor and the orexin-2 receptor. The term includes suvorexant (sold as Belsomra (servorexant) by Merck), daridorexant (sold as Quvivic (daridorexant) by Idorsia) and Lemborexant (sold as Dayvigo (lemborexant) by Eisai Pharmaceuticals).

As used herein, the term “food related disorder” includes any disorder in a human that is associated with eating or not eating food. The disorder may occur in a human of any weight, including underweight humans, overweight humans, and humans of normal weight. The term includes, but is not limited to, binge eating disorder, obesity, overweight, bulimia nervosa, food addiction (defined by the Yale Food Addiction Scale; YFAS), excessive weight, Prader Willi Syndrome, or Other Specified Feeding or Eating Disorder (OSFED).

The term “animal” includes mammals, fish, amphibians, reptiles, birds and invertebrates. The term “mammal” includes humans, higher non-human primates, rodents, domestic, cows, horses, pigs, sheep, dogs and cats. In one embodiment, the animal is a mammal. In one embodiment, the animal is a human. The term “patient” as used herein refers to any animal including mammals. In one embodiment, the patient is a mammalian patient. In one embodiment, the patient is a human patient.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

Specific values listed below are for illustration only; they do not exclude other defined values or other values within defined ranges for the radicals and substituents. It is to be understood that two or more values may be combined. It is also to be understood that the values listed herein below (or subsets thereof) can be excluded.

In one embodiment, the amphetamine is levoamphetamine or a pharmaceutically acceptable salt thereof or a prodrug thereof.

In one embodiment, the amphetamine is dextroamphetamine or a pharmaceutically acceptable salt thereof or a prodrug thereof.

In one embodiment, the 0.40 mg/kg of the amphetamine and the orexin receptor antagonist are administered to the human once daily.

In one embodiment, the administration of the amphetamine and the orexin receptor antagonist results in less sleep disturbance than the administration of the amphetamine alone.

In one embodiment, the administration of the amphetamine and the orexin receptor antagonist results in less insomnia, sleep fragmentation, wakefulness, or sleep disturbances than the administration of the amphetamine alone.

In one embodiment, the administration of the stimulant and the orexin receptor antagonist results in less insomnia, sleep fragmentation, wakefulness, or sleep disturbances than the administration of the amphetamine alone.

In one embodiment, the administration of the amphetamine and the orexin receptor antagonist results in less insomnia than the administration of the amphetamine alone.

In one embodiment, less than about 0.30 mg/kg of the amphetamine is administered.

In one embodiment, less than about 0.20 mg/kg of the amphetamine) is administered.

In one embodiment, less than about 0.10 mg/kg of the amphetamine) is administered.

In one embodiment, less than about 0.05 mg/kg of the amphetamine is administered.

In one embodiment, less than about 0.04 mg/kg of the amphetamine is administered.

In one embodiment, less than about 0.03 mg/kg of the amphetamine is administered.

In one embodiment, less than about 0.02 mg/kg of the amphetamine is administered.

In one embodiment, the orexin receptor antagonist is a selective orexin-1 receptor antagonist or a selective orexin-2 receptor antagonist.

In one embodiment, the orexin receptor antagonist is a dual orexin receptor antagonist.

In one embodiment, the dual orexin receptor antagonist is suvorexant, daridorexant, or lemborexant.

In one embodiment, the dual orexin receptor antagonist is suvorexant.

In one embodiment, less than about 25 mg suvorexant is administered to the human daily.

In one embodiment, less than about 20 mg suvorexant is administered.

In one embodiment, less than about 15 mg suvorexant is administered.

In one embodiment, less than about 10 mg suvorexant is administered.

In one embodiment, less than about 5 mg suvorexant is administered.

In one embodiment, less than about 4 mg suvorexant is administered.

In one embodiment, less than about 3 mg suvorexant is administered.

In one embodiment, less than about 2 mg suvorexant is administered.

In one embodiment, less than about 1 mg suvorexant is administered.

In one embodiment, the food related disorder is a condition associated with hyperphagia.

In one embodiment, the food related disorder is binge eating disorder, obesity, overweight, bulimia nervosa, food addiction (defined by the Yale Food Addiction Scale; YFAS). excessive weight, Prader Willi Syndrome, or Other Specified Feeding or Eating Disorder (OSFED).

In one embodiment, the food related disorder is binge eating disorder. In one embodiment, the pharmaceutical composition is formulated for oral administration.

In one embodiment, the pharmaceutical composition is formulated as a tablet or a capsule.

In one embodiment, the pharmaceutical composition comprises less than about 30 mg of amphetamine.

In one embodiment, the pharmaceutical composition comprises less than about 20 mg of amphetamine.

In one embodiment, the pharmaceutical composition comprises less than about 10 mg of amphetamine.

In one embodiment, the pharmaceutical composition comprises less than about 5 mg of amphetamine.

In one embodiment, the pharmaceutical composition comprises less than about 3 mg of amphetamine.

In one embodiment, the pharmaceutical composition comprises less than about 25 mg of the orexin receptor antagonist.

In one embodiment, the pharmaceutical composition comprises less than about 15 mg of the orexin receptor antagonist.

In one embodiment, the pharmaceutical composition comprises less than about 10 mg of the orexin receptor antagonist.

In one embodiment, the pharmaceutical composition comprises less than about 5 mg of the orexin receptor antagonist.

In one embodiment, the amphetamine comprises d-amphetamine or a salt thereof.

In one embodiment, the administration of the amphetamine and the orexin receptor antagonist together results in less abuse liability than the administration of the amphetamine alone. The reduction in abuse liability results from the lower dose of amphetamine required to achieve the desired therapeutic effect when the amphetamine is administered in combination with the orexin receptor antagonist, as well as anti-addiction properties of the orexin receptor antagonist.

### Methods for Lowering the Amount of Amphetamine Required to Achieve a Therapeutic Result

In one embodiment, the invention provides a method for lowering the amount of an amphetamine required to achieve a therapeutic result in a human, comprising, administering the amphetamine to the human in combination with an orexin receptor antagonist.

Currently a 60 mg dose of d-Amphetamine is approved for the treatment of narcolepsy and a 40 mg dose of d-Amphetamine is approved for the treatment of ADHD; a maximum 70 mg dose of Lisdexamfetamine is approved for use; a 50 mg dose of a mixed salts of a single-entity amphetamine product (marketed as Mydayis) is approved for use; and a 200 mg dose of modafinil is approved for use. These agents can also be administered at lower dosages depending on the patient and the condition to be treated.

The method of the invention allows the dosage of the amphetamine to be lowered in a given patient, whilst still achieving a similar therapeutic result, by administering the amphetamine in combination with an orexin receptor inhibitor. In one embodiment, the amount of the amphetamine is lowered by 40%. In one embodiment, the amount of the amphetamine is lowered by 50%. In one embodiment, the amount of the amphetamine is lowered by 60%. In one embodiment, the amount of the amphetamine is lowered by 70%. In one embodiment, the amount of the amphetamine is lowered by 80%. In one embodiment, the amount of the amphetamine is lowered by 90%. In one embodiment, the side effects associated with the administration of the amphetamine are reduced by lowering the effective dose amphetamine that is administered.

In one embodiment, the dose of d-Amphetamine is reduced to less than about 50 mg, 40 mg, 30 mg, 20 mg, 10 mg, 5 mg, 3 mg, 2 mg, 1 mg, 0.5 mg, 0.3 mg, or 0.1 mg.

In one embodiment, the dose of lisdexamfetamine is reduced to less than about 60 mg, 50 mg, 40 mg, 30 mg, 20 mg, 10 mg, 5 mg, 3 mg, 2 mg, 1 mg, 0.5 mg, 0.3 mg, or 0.1 mg.

In one embodiment, the dose of mixed salts of a single-entity amphetamine product (marketed as Mydayis) is reduced to less than about 37.5 mg, 25 mg, 12.5 mg, 10 mg, 7.5 mg, 5 mg, 3 mg, 2 mg, 1 mg, 0.5 mg, 0.3 mg, or 0.1 mg.

In one embodiment, the dose of modafinil is reduced to less than about 100 mg, 50 mg, 30 mg, 10 mg, 5 mg, 3 mg, 2 mg, or 1 mg.

Suvorexant is currently approved at a maximum dose of 20 mg. In the methods and compositions of the invention, either a higher or a lower dose of suvorexant can be used. For example, in one embodiment, the dose of suvorexant is less than about 40 mg, 30 mg, 20 mg, 15 mg, 10 mg, 5 mg, 3 mg, 2 mg, 1 mg, 0.5 mg, 0.3 mg, 0.1 mg.

Daridorexant is currently approved at a maximum dose of 50 mg. In the methods and compositions of the invention, either a higher or a lower dose of daridorexant can be used. For example, in one embodiment, the dose of daridorexant is less than about 70 mg, 60 mg, 50 mg, 25 mg, 20 mg, 10 mg, 5 mg, 3 mg, 2 mg, 1 mg, 0.5 mg, 0.3 mg, 0.1 mg.

Lemborexant is currently approved at a maximum dose of 10 mg. In the methods and compositions of the invention, either a higher or a lower dose of lemborexant can be used. For example, in one embodiment, the dose of Lemborexant is less than about 30 mg, 20 mg, 10 mg, 5 mg, 3 mg, 2 mg, 1 mg, 0.5 mg, 0.3 mg, 0.1 mg

Administration of a compound as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Compounds can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention will now be illustrated by the following non-limiting Examples.

### EXAMPLES

### Example 1

### The Combination of Low Doses of d-Amphetamine and Suvorexant Reduces Palatable Food Intake

Several health conditions are associated with the overconsumption of food, including obesity, overweight, binge eating disorder, bulimia nervosa, food addiction, and other specified feeding or eating disorders (Guerdjikova A I et al., Medical Clin North Am, 2019, 103(40): 669-680; Schulte E M et al., Psych Addict Behav, 2019, 33(2): 144-153). d-amphetamine and associated compounds have anorectic properties, however these compounds have high abuse liability and off-target effects, including disruption of sleep, at doses that are necessary to suppress food intake (McElroy S L et al., Neuropsychopharm, 2016, 41(5):1251-60); Jasinksi D R & Krishnan S, J Psychopharmacol, 2009, 23(4): 419-27). Here, the effect of combining subclinical doses of d-amphetamine with the dual orexin receptor antagonist suvorexant on feeding outcomes was examined in rats.

Rats were trained to consume a palatable fat mixture (90% vegetable shortening, 10% sucrose) during brief exposure periods (30 mins) that occurred twice per week. The amount of palatable food consumed during these 30 min exposure periods was measured.

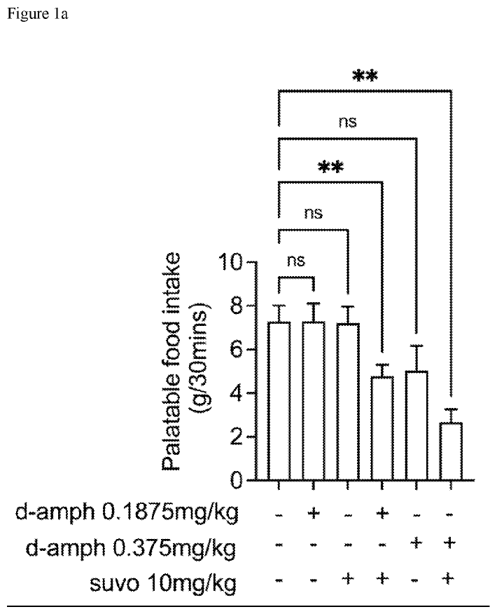

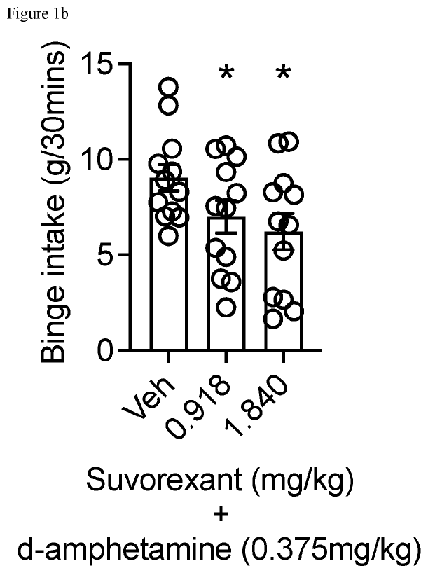

FIG. 1a shows that when rats were treated with low doses of d-amphetamine (0.1875, 0.375 mg/kg; i.p) prior to test sessions, there was no significant change in palatable food intake. Similarly, when rats were treated with suvorexant (10 mg/kg; i.p.) prior to test sessions, there was no significant change in palatable food intake. When low doses of d-amphetamine (0.1875, 0.375 mg/kg; i.p) and suvorexant (10 mg/kg; i.p.) were co-administered prior to test sessions, there was a significant reduction in palatable food intake. This indicates synergistic effects of combining d-amphetamine and suvorexant in the suppression of food intake. RM ANOVA F(3.25, 35.73)=9.73, p<0.0001. Holm-Sidak post-hoc tests: **p<0.05. ns: p>0.05.

In FIG. 1a, ‘+’ indicates that compound was administered, whereas ‘−’ indicates that no compound or its vehicle was administered. N=12 for all treatment groups (within-subjects design; compounds and their combination administered in a counterbalanced manner).

### Example 2

FIG. 1b shows that when a low dose of d-amphetamine (0.375 mg/kg; i.p) was co-administered with low doses of suvorexant (0.918 and 1.84 mg/kg; i.p.) prior to test sessions, there was a significant reduction in palatable food intake. This indicates synergistic effects of combining d-amphetamine and suvorexant in the suppression of food intake.

### Example 3

### The Combination of Low Doses of d-Amphetamine and SB334867 Reduces Palatable Food Intake

Rats were trained to consume a palatable fat mixture (90% vegetable shortening, 10% sucrose) during brief exposure periods (30 mins) that occurred twice per week. The amount of palatable food consumed during these 30 min exposure periods was measured.

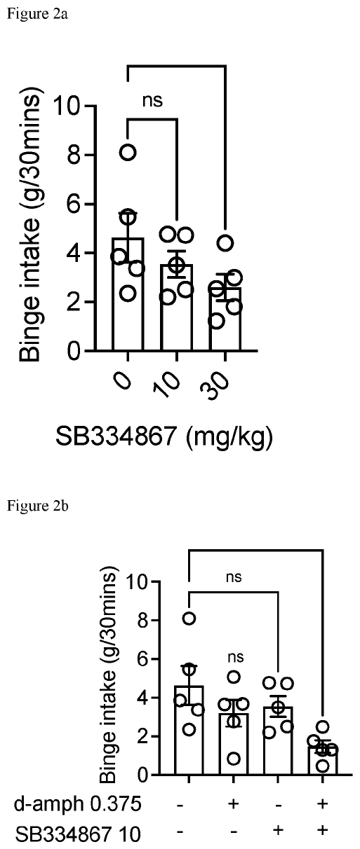

FIG. 2a shows that when rats were treated with SB334867 (10, 30 mg/kg; i.p.) prior to test sessions, a significant change in palatable food intake was observed only at the highest dose (30 mg/kg; i.p).

FIG. 2b shows that when a low dose of d-amphetamine (0.375 mg/kg; i.p) and a low dose of SB334867 (10 mg/kg; i.p.) were co-administered prior to test sessions, there was a significant reduction in palatable food intake. This indicates synergistic effects of combining d-amphetamine and SB334867 in the suppression of food intake.

In FIG. 2b, ‘+’ indicates that compound was administered, whereas ‘−’ indicates that no compound or its vehicle was administered. N=5 for all treatment groups (within-subjects design; compounds and their combination administered in a counterbalanced manner).

Data for Example 3 is provided in FIGS. 2a-2b. Concurrent treatment with Ox1R antagonist and d-amphetamine (at doses that are ineffective on their own) reduces binge-like eating.

### Example 4

### Suvorexant Reduces Lever Responding for Food

Heightened motivation for food is characteristic of several health conditions associated with the overconsumption of food, including obesity, overweight, binge eating disorder, bulimia nervosa, food addiction, and other specified feeding or eating disorders (https://pubmed.ncbi.nlm.nih.gov/33905755/). In rats, operant responding for food is commonly used to determine motivation for food (https://pubmed.ncbi.nlm.nih.gov/36623582/). The combination of low doses of d-amphetamine and suvorexant, found in Experiment 1 to reduce palatable food intake, also tested to determine if it reduced rats' willingness to lever respond for a food reward.

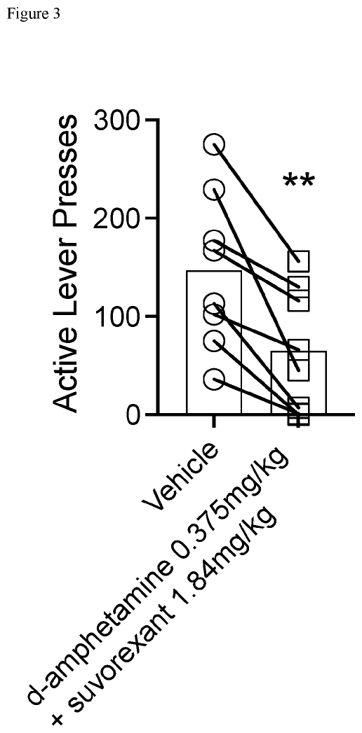

Rats were trained to lever press for sucrose pellets on a fixed ratio (FR) 1 schedule, meaning that every lever press was rewarded with a sucrose pellet. FIG. 3 shows that when a low dose of d-amphetamine (0.375 mg/kg; i.p) was co-administered with a low dose of suvorexant (1.84 mg/kg; i.p.) prior to test sessions, there was a significant reduction in lever responding for food reward. This indicates at combination doses that reduce palatable food intake (Example 1), low doses of d-amphetamine and suvorexant also reduce general motivation for food (relevant to obesity, overweight, binge eating disorder, bulimia nervosa, ‘food addiction’).

### Example 5

Repeated exposure to psychostimulants results in behavioral sensitization, characterized by an augmented locomotor response to subsequent injections of psychostimulants (Pierce & Kalivas, 1997; sciencedirect.com/science/article/pii/S0165017397000210). These behavioral changes reflect neurochemical changes observed in ‘addiction’, and thus can be used as a proxy of a drug's abuse liability (Wearne & Cornish, 2019; sciencedirect.com/science/article/pii/S0278584619303112?via%3Dihub). The co-administration of d-amphetamine and an orexin receptor was tested to determine if the antagonist abrogated the development of behavioral sensitization.

Rats were measured for baseline locomotor activity (Day 0). Rats then received injections of saline, d-amphetamine, or the combination of d-amphetamine and suvorexant, for 7 consecutive days. Locomotor activity was recorded for 1 hour following treatment.

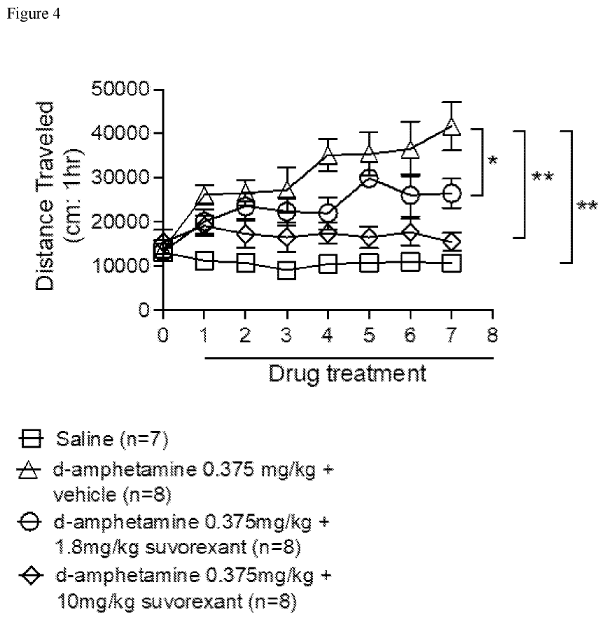

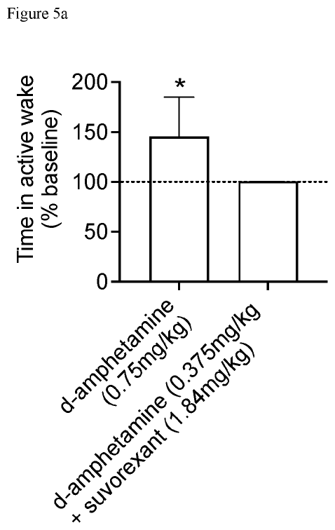

FIG. 4 shows that rats from all treatment groups had similar locomotor activity at baseline. On the final treatment day (day 7), behavioral reactivity was significantly higher in rats treated with d-amphetamine alone compared to those treated with d-amphetamine (0.375 mg/kg) combined with low doses of suvorexant (1.8, 10 mg/kg). These data indicate that the combination of d-amphetamine and low doses of suvorexant blocks behavioral sensitization normally seen in response to repeated exposure to d-amphetamine. By extension, these data indicate that the combination of d-amphetamine with low doses of suvorexant abrogates the abuse liability of d-amphetamine. They also indicate that the combination of d-amphetamine and suvorexant abrogates the activity stimulating properties of d-amphetamine.

### Example 6

### Co-Administration of an Orexin Receptor 1/2 Antagonist With d-Amphetamine Reduces the Sleep-Disturbing Effects Normally Observed With d-Amphetamine Treatment

Psychostimulants, including d-amphetamine, promote wakefulness and disturb sleep (Rechtschaffen & Maron, 1964 (sciencedirect.com/science/article/pii/0013469464900860); Saletu et al., 1989 (pubmed.ncbi.nlm.nih.gov/2568348)). The combination of low doses of d-amphetamine and an orexin 1/2 receptor antagonist (suvorexant) were tested to determine if they could abrogate the sleep disturbing effects of d-amphetamine administered alone.

Rats were implanted with a telemetry device to wirelessly collect electroencephalography (EEG) and electromyography (EMG) signals over the course of the experiment. Prior to any treatment, EEG and EMG signals were measured over the 12 hours inactive period to determine time spent in different stages of sleep at baseline. Rats were then treated with d-amphetamine (0.75 mg/kg; i.p.), which is the dose necessary to suppress palatable food intake when administered alone, or low doses of d-amphetamine (0.375 mg/kg; i.p.) and suvorexant (1.84 mg/kg; i.p.) in combination. Injections were made approximately 1 hour prior to the onset of the inactive (lights on) period.

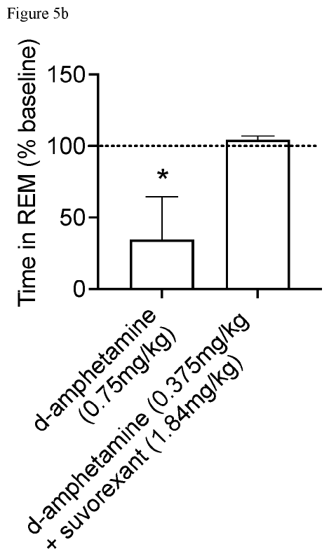

FIG. 5a shows that compared to baseline, d-amphetamine alone (0.75 mg/kg) increased the amount of time spent in active wake. In contrast, the combination of low doses of d-amphetamine (0.375 mg/kg) and suvorexant (1.84 mg/kg) did not affect time spent in active wake. FIG. 5b shows that compared to baseline, d-amphetamine alone (0.75 mg/kg) decreased the amount of time spent in rapid eye movement (REM) sleep. In contrast, the combination of low doses of d-amphetamine (0.375 mg/kg) and suvorexant (1.84 mg/kg) had no effect on time spent in REM. Together, these data indicate that co-administration of Ox1R/Ox2R antagonist with d-amphetamine reduces the wake-promoting effects normally observed with d-amphetamine treatment.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. (canceled)

2. The method of claim 76, wherein the disease is a food related disorder in a human and wherein less than about 0.40 mg/kg of the amphetamine and the orexin receptor antagonist are administered to the human.

3. (canceled)

4. The method of claim 2, wherein the amphetamine is dextroamphetamine or a pharmaceutically acceptable salt thereof or a prodrug thereof.

5-9. (canceled)

10. The method of claim 2, wherein less than about 0.20 mg/kg of the amphetamine) is administered.

11-18. (canceled)

19. The method of claim 2, wherein the orexin receptor antagonist is suvorexant.

20. The method of claim 19, wherein less than about 25 mg suvorexant is administered to the human daily.

21-25. (canceled)

26. The method of claim 2, wherein the food related disorder is binge eating disorder, obesity, overweight, bulimia nervosa, food addiction, excessive weight, Prader Willi Syndrome, or Other Specified Feeding or Eating Disorder (OSFED).

27-29. (canceled)

30. A pharmaceutical composition for once daily dosing for the treatment of a food related disorder in a human, comprising less than about 40 mg of an amphetamine, an orexin receptor antagonist, and a pharmaceutically acceptable carrier.

31-34. (canceled)

35. The pharmaceutical composition of claim 30, which comprises less than about 10 mg of amphetamine.

36-40. (canceled)

41. The pharmaceutical composition of claim 30, wherein the orexin receptor antagonist is suvorexant.

42. The pharmaceutical composition of claim 30, that comprises less than about 25 mg of the orexin receptor antagonist.

43-46. (canceled)

47. The pharmaceutical composition of claim 30, wherein the food related disorder is binge eating disorder, obesity, overweight, bulimia nervosa, food addiction, excessive weight, Prader Willi Syndrome, or Other Specified Feeding or Eating Disorder (OSFED).

48. (canceled)

49. The pharmaceutical composition of claim 30 that comprises less than about 3 mg of amphetamine, less than about 5 mg suvorexant, and a pharmaceutically acceptable carrier.

50. (canceled)

51. A method for lowering the amount of an amphetamine required to achieve a therapeutic result in a human, comprising, administering the amphetamine to the human in combination with an orexin receptor antagonist.

52. The method of claim 51, wherein the amphetamine comprises d-amphetamine or a salt thereof.

53. The method of claim 51, wherein the amount of the amphetamine is lowered by at least about 40%.

54-58. (canceled)

59. The method of claim 51, wherein the orexin receptor antagonist is suvorexant.

60. The method of claim 51, wherein the therapeutic result is the treatment of a food related disorder.

61. (canceled)

62. The method of claim 60, wherein the food related disorder is binge eating disorder, obesity, overweight, bulimia nervosa, food addiction, excessive weight, Prader Willi Syndrome, or Other Specified Feeding or Eating Disorder (OSFED).

63-73. (canceled)

74. The method of claim 2 wherein the administration of the amphetamine and the orexin receptor antagonist results in less abuse liability than the administration of the amphetamine alone.

75. (canceled)

76. A method comprising, treating a disease that is treatable with a stimulant in an animal by administering to the animal, the stimulant and an orexin receptor antagonist that reduces the abuse liability, wake promoting or other unwanted side effect of the stimulant.

77-81. (canceled)
